(19) United States
(12) Patent Application Publication
(10) Pub. No.: US 2011/0189149 A1
(43) Pub. Date: Aug. 4, 2011

(54) New Uses of Lactic Acid Bacteria and Bifidobacteria

(76) Inventors: Remy Burcelin, Escalquens (FR); Didier Carcano, Paris (FR); Pierre Desreumaux, Mouvaux (FR); Sampo Lahtinen, Siuntio (FI)

(21) Appl. No.: 12/999,555

(22) PCT Filed: Jun. 20, 2008
(86) PCT No.: PCT/IB09/06295

§ 371 Date: Apr. 26, 2011

(51) Int. Cl. A61K 35/74; A61P 3/00; A61P 9/00; A61P 29/00

(52) U.S. Cl. 424/93.45

(57) ABSTRACT

The invention comprises use of a bacterium selected from a lactic acid bacterium, a Bifidobacterium and a mixture of any thereof in the manufacture of a food product, dietary supplement or medicament for lowering tissue inflammation (in particular, adipose, liver or muscle tissue inflammation) and treating cardiovascular disease in a mammal. The lactic acid bacteria and/or Bifidobacteria can also be used to treat diabetes and insulin resistance and induce enhanced secretion of insulin upon a glucose challenge and lower blood glucose levels without a concomitant decrease in weight gain.

### FIELD OF THE INVENTION

This invention relates to new uses of lactic acid bacteria and Bifidobacteria, (particularly, although not exclusively, probiotic bacteria), and to food products, feed products, dietary supplements and pharmaceutical formulations containing them.

### DESCRIPTION OF THE PRIOR ART

Diabetes mellitus, often referred to simply as diabetes, is a condition characterized by disordered metabolism and abnormally high blood sugar (hyperglycaemia) resulting from insufficient levels and/or action of the hormone insulin. The characteristic symptoms are excessive urine production (polyuria) due to high blood glucose levels, excessive thirst and increased fluid intake (polydipsia) attempting to compensate for increased urination, blurred vision due to high blood glucose effects on the eye's optics, unexplained weight loss, and lethargy. These symptoms are likely to be less apparent if the blood sugar is only mildly elevated.

The World Health Organisation recognises three main forms of diabetes mellitus: type 1, type 2, and gestational diabetes (occurring during pregnancy), which have different causes and population distributions. While, ultimately, all forms are due to the beta cells of the pancreas being unable to produce sufficient insulin to prevent hyperglycemia, the causes are different. Type 1 diabetes is usually due to autoimmune destruction of the pancreatic beta cells. Type 2 diabetes is characterized by insulin resistance in target tissues. This causes a need for abnormally high amounts of insulin and diabetes develops when the beta cells cannot meet this demand. Gestational diabetes is similar to type 2 diabetes in that it involves insulin resistance; the hormones of pregnancy can cause insulin resistance in women genetically predisposed to developing this condition.

Gestational diabetes typically resolves with delivery of the child: however, types 1 and 2 diabetes are chronic conditions. All types have been treatable since insulin became medically available in 1921. Type 1 diabetes, in which insulin is not secreted by the pancreas, is directly treatable only with injected insulin, although dietary and other lifestyle adjustments are part of management. Type 2 may be managed with a combination of dietary treatment, tablets and injections and, frequently, insulin supplementation.

Diabetes can cause many complications. Acute complications (hypoglycemia, ketoacidosis or nonketotic hyperosmolar coma) may occur if the disease is not adequately controlled. Serious long-term complications include cardiovascular disease (doubled risk), chronic renal failure, retinal damage (which can lead to blindness), nerve damage (of several kinds), and microvascular damage, which may cause impotence and poor healing. Poor healing of wounds, particularly of the feet, can lead to gangrene, which may require amputation. Adequate treatment of diabetes, as well as increased emphasis on blood pressure control and lifestyle factors (such as not smoking and keeping a healthy body weight), may improve the risk profile of most aforementioned complications. In the developed world, diabetes is the most significant cause of adult blindness in the non-elderly and the leading cause of non-traumatic amputation in adults, and diabetic nephropathy is the main illness requiring renal dialysis in the United States.

Diabetes mellitus is currently a chronic disease, without a cure, and medical emphasis must necessarily be on managing/avoiding possible short-term as well as long-term diabetes-related problems. There is an exceptionally important role for patient education, dietetic support, sensible exercise, self glucose monitoring, with the goal of keeping both short-term blood glucose levels, and long term levels as well, within acceptable bounds. Careful control is needed to reduce the risk of long term complications. This is theoretically achievable with combinations of diet, exercise and weight loss (type 2), various oral diabetic drugs (type 2 only), and insulin use (type 1 and increasingly for type 2 not responding to oral medications). In addition, given the associated higher risks of cardiovascular disease, lifestyle modifications should be undertaken to control blood pressure and cholesterol by exercising more, smoking cessation, consuming an appropriate diet, wearing diabetic socks, and if necessary, taking any of several drugs to reduce pressure.

Oral antidiabetic drugs and insulin analogs currently on the market or undergoing clinical trials include biguanides (such as metformin), sulfonylureas (such as carbutamide, chlorpropamide, glibenclamide (Glyburide), gliclazide, glimepiride, glipizide, gliquidone, tolazamide or tolbutamide), alpha-glucosidase inhibitors (such as acarbose, miglitol or voglibose), thiazolidinediones (TZD) (such as pioglitazone, rivoglitazone or rosiglitazone), meglitinides (such as nateglinide, repaglinide or mitiglinide), dipeptidyl peptidase-4 (DPP-4) inhibitors (such as alogliptin, saxagliptin, sitagliptin or vildagliptin), glucagon-like peptide-1 analogs (such as exenatide, liraglutide, or albiglutide), amylin analogs (such as pramlintide), fast acting insulin analogs (such as insulin lispro, insulin aspart and insulin glulisine), long acting insulin analogs (such as insulin glargine, insulin detemir), dual PPAR agonists (such as aleglitazar) and SGLT2 inhibitors (such as dapagliflozin, remogliflozin and sergliflozin).

Type 2 diabetes is often associated with obesity. The body mass index (BMI) (calculated as weight in kilograms divided by the square of height in metres) is the most commonly accepted measurement for overweight and/or obesity. A BMI exceeding 25 is considered overweight. Obesity is defined as a BMI of 30 or more, with a BMI of 35 or more considered as serious comorbidity obesity and a BMI of 40 or more considered morbid obesity. Mortality is increased in obesity, with a BMI of over 32 being associated with a doubled risk of death. There are alterations in the body's response to insulin (insulin resistance), a proinflammatory state and an increased tendency to thrombosis (prothrombotic state).

Central obesity (male-type or waist-predominant obesity, characterised by a high waist-hip ratio), is a particularly important risk factor for diabetes and metabolic syndrome, the clustering of a number of diseases and risk factors that heavily predispose for cardiovascular disease. These are diabetes mellitus type 2, high blood pressure, high blood cholesterol, and triglyceride levels (combined hyperlipidemia).

The use of microorganisms in treating obesity, diabetes and diabetes-related conditions is in general known in the art. For example, WO 02/38165 describes use of a strain of Lactobacillus (in particular, Lactobacillus plantarum) in reducing the risk factors involved in the metabolic syndrome.

WO 2007/043933 describes the use of probiotic bacteria (in particular, Lactobacillus casei F19, Lactobacillus acidophilus NCFB 1748 or Bifidobacterium lactis Bb12) for the manufacture of food and feed products, dietary supplements, for controlling weight gain, preventing obesity, increasing satiety, prolonging satiation, reducing food intake, reducing fat deposition, improving energy metabolism, enhancing insulin sensitivity, treating obesity and treating insulin insensitivity.

US 2002/0037577 describes the use of microorganisms, such as Lactobacilli, for the treatment or prevention of obesity or diabetes mellitus by reduction of the amount of monosaccharide or disaccharide which may be absorbed into the body, by converting such compounds into polymeric materials which cannot be absorbed by the intestine. However, in this document the obesity in the mice is genetically induced, resulting from a leptin deficiency which occurs extremely rarely in humans. Therefore, the results obtained in this document would not be considered relevant to diet-induced obesity, diabetes and metabolic diseases since plasma leptin concentration is increased in human and nutrient-induced metabolic diseases.

### SUMMARY OF THE INVENTION

In one aspect, the invention comprises use of a bacterium selected from a lactic acid bacterium, a Bifidobacterium or a mixture of any thereof in the manufacture of a food product, dietary supplement or medicament for lowering adipose tissue inflammation in a mammal.

In another aspect, the invention comprises use of a bacterium selected from a lactic acid bacterium, a Bifidobacterium or a mixture of any thereof in the manufacture of a food product, dietary supplement or medicament for treating cardiovascular disease in a mammal.

In a further aspect, the invention comprises use of a bacterium selected from a lactic acid bacterium, a Bifidobacterium or a mixture of any thereof in the manufacture of a food product, dietary supplement or medicament for reducing the blood glucose level in a diabetic and/or obese mammal without a concomitant decrease in weight gain.

In a still further aspect, the invention comprises use of a bacterium selected from a lactic acid bacterium, a Bifidobacterium or a mixture of any thereof in the manufacture of a food product, dietary supplement or medicament for reducing insulin resistance in a diabetic and/or obese mammal without a concomitant decrease in weight gain.

In a yet further aspect, the invention comprises use of a bacterium selected from a lactic acid bacterium, a Bifidobacterium or a mixture of any thereof in the manufacture of a food product, dietary supplement or medicament for treating diabetes in a mammal without a concomitant decrease in weight gain.

In a still further aspect, the invention comprises use of a bacterium selected from a lactic acid bacterium, a Bifidobacterium or a mixture of any thereof in the manufacture of a food product, dietary supplement or medicament for decreasing the metabolic consequences of diabetes in a diabetic and, optionally, obese mammal.

In a yet further aspect, the invention comprises use of a bacterium selected from a lactic acid bacterium, a Bifidobacterium or a mixture of any thereof in the manufacture of a food product, dietary supplement or medicament for improving glucose tolerance in a mammal.

In a yet further aspect, the invention comprises use of a bacterium selected from a lactic acid bacterium, a Bifidobacterium or a mixture of any thereof in the manufacture of a food product, dietary supplement or medicament for improving glucose tolerance in a diabetic and/or obese mammal.

In a still further aspect, the invention comprises use of a lactic acid bacterium or a mixture thereof in the manufacture of a food product, dietary supplement or medicament for increasing fed insulin secretion in a mammal.

In a yet further aspect, the invention comprises use of a lactic acid bacterium or a mixture thereof in the manufacture of a food product, dietary supplement or medicament for lowering liver tissue inflammation in a mammal.

In a still further aspect, the invention comprises use of a lactic acid bacterium or a mixture thereof in the manufacture of a food product, dietary supplement or medicament for lowering muscle tissue inflammation in a mammal.

In another aspect, the invention comprises a bacterium selected from a lactic acid bacterium, a Bifidobacterium or a mixture of any thereof for use in lowering adipose tissue inflammation in a mammal.

In another aspect, the invention comprises a bacterium selected from a lactic acid bacterium, a Bifidobacterium or a mixture of any thereof for use in treating cardiovascular disease in a mammal.

In a further aspect, the invention comprises a bacterium selected from a lactic acid bacterium, a Bifidobacterium or a mixture of any thereof for use in reducing the blood glucose level in a diabetic and/or obese mammal without a concomitant decrease in weight gain.

In a still further aspect, the invention comprises a bacterium selected from a lactic acid bacterium, a Bifidobacterium or a mixture of any thereof for use in reducing insulin resistance and/or at least one consequence thereof in a diabetic and/or obese mammal without a concomitant decrease in weight gain.

In a yet further aspect, the invention comprises a bacterium selected from a lactic acid bacterium, a Bifidobacterium or a mixture of any thereof for use in treating diabetes and/or at least one complication thereof in a mammal without a concomitant decrease in weight gain.

In a still further aspect, the invention comprises a bacterium selected from a lactic acid bacterium, a Bifidobacterium or a mixture of any thereof for use in decreasing the metabolic consequences of diabetes in a diabetic and, optionally, obese mammal.

In a yet further aspect, the invention comprises a bacterium selected from a lactic acid bacterium, a Bifidobacterium or a mixture of any thereof for use in improving glucose tolerance in a mammal.

In a yet further aspect, the invention comprises a bacterium selected from a lactic acid bacterium, a Bifidobacterium or a mixture of any thereof for use in improving glucose tolerance in a diabetic and/or obese mammal.

In a still further aspect, the invention comprises a lactic acid bacterium or a mixture thereof for use in increasing fed insulin secretion in a mammal.

In a yet further aspect, the invention comprises a lactic acid bacterium or a mixture thereof for use in lowering liver tissue inflammation in a mammal.

In a still further aspect, the invention comprises a lactic acid bacterium or a mixture thereof for use in lowering muscle tissue inflammation in a mammal.

In another aspect, the invention comprises a method of lowering adipose tissue inflammation in a mammal, comprising administering to the mammal an effective amount of a bacterium selected from a lactic acid bacterium, a Bifidobacterium or a mixture of any thereof.

In another aspect, the invention comprises a method of treating cardiovascular disease in a mammal, comprising administering to the mammal an effective amount of a bacterium selected from a lactic acid bacterium, a Bifidobacterium or a mixture of any thereof.

In a further aspect, the invention comprises a method of reducing the blood glucose level in a diabetic and/or obese mammal without a concomitant decrease in weight gain, comprising administering to the mammal an effective amount of a bacterium selected from a lactic acid bacterium, a Bifidobacterium or a mixture of any thereof.

In a still further aspect, the invention comprises a method of reducing insulin resistance and/or at least one consequence thereof in a diabetic and/or obese mammal without a concomitant decrease in weight gain, comprising administering to the mammal an effective amount of a bacterium selected from a lactic acid bacterium, a Bifidobacterium or a mixture of any thereof.

In a yet further aspect, the invention comprises a method of treating diabetes and/or at least one complication thereof in a mammal without a concomitant decrease in weight gain, comprising administering to the mammal an effective amount of a bacterium selected from a lactic acid bacterium, a Bifidobacterium or a mixture of any thereof.

In a yet further aspect, the invention comprises a method of decreasing the metabolic consequences of diabetes in a diabetic and, optionally, obese mammal, comprising administering to the mammal an effective amount of a bacterium selected from a lactic acid bacterium, a Bifidobacterium or a mixture of any thereof.

In a yet further aspect, the invention comprises a method of improving glucose tolerance in a mammal, comprising administering to the mammal an effective amount of a bacterium selected from a lactic acid bacterium, a Bifidobacterium or a mixture of any thereof.

In a yet further aspect, the invention comprises a method of improving glucose tolerance in a diabetic and/or obese mammal, comprising administering to the mammal an effective amount of a bacterium selected from a lactic acid bacterium, a Bifidobacterium or a mixture of any thereof.

In a still further aspect, the invention comprises a method of increasing fed insulin secretion in a mammal, comprising administering to the mammal an effective amount of a lactic acid bacterium or a mixture thereof.

In a yet further aspect, the invention comprises a method of lowering muscle tissue inflammation in a mammal, comprising administering to the mammal an effective amount of a lactic acid bacterium or a mixture thereof.

In a still further aspect, the invention comprises a method of lowering liver tissue inflammation in a diabetic and/or obese mammal, comprising administering to the mammal an effective amount of a lactic acid bacterium or a mixture thereof.

### BRIEF DESCRIPTION OF THE DRAWINGS

FIGS. 1a and 1b illustrate the plasma glucose levels made during the IPIST test in diabetic and obese mice and in control mice before Lactobacillus acidophilus NCFM treatment;

FIG. 2 illustrates the fasting plasma glucose levels in diabetic and obese mice and in control mice before and after Lactobacillus acidophilus NCFM treatment;

FIG. 3 illustrates the weight gain in obese and control mice in the same study;

FIG. 4a illustrates the effect of Lactobacillus acidophilus NCFM treatment on the intensity of subcutaneous adipose tissue M1 marker in mice fed with a high-fat diet (HFD);

FIG. 4b illustrates the effect of Lactobacillus acidophilus NCFM treatment on the intensity of subcutaneous adipose tissue M2 marker in mice fed with HFD;

FIG. 4c illustrates the effect of Lactobacillus acidophilus NCFM treatment on the intensity of subcutaneous adipose tissue markers M1 and M2 in mice fed with HFD; and

FIG. 4d illustrates the effect of Lactobacillus acidophilus NCFM treatment on the intensity of other subcutaneous adipose tissue markers in mice fed with HFD.

### DETAILED DESCRIPTION OF THE INVENTION

### Lactic Acid Bacteria and Bifidobacteria

The bacterium used in embodiments of the present invention is selected from a lactic acid bacterium (LAB), a Bifidobacterium or a mixture of any thereof. In this specification the term ‘lactic acid bacterium’ includes any bacterium capable of producing, as the major metabolic end product of carbohydrate fermentation, lactic acid or at least one of its derivatives (including, but not limited to, acetic acid or propionic acid): the term is therefore intended to include propionic acid bacteria (PAB), which produce propionic acid as a carbohydrate fermentation product.

The bacterium may be used in any form capable of exerting the effects described herein. For example, the bacteria may be viable, dormant, inactivated or dead bacteria. Preferably, the bacteria are viable bacteria.

The bacteria may comprise whole bacteria or may comprise bacterial components. Examples of such components include bacterial cell wall components such as peptidoglycan, bacterial nucleic acids such as DNA and RNA, bacterial membrane components, and bacterial structural components such as proteins, carbohydrates, lipids and combinations of these such as lipoproteins, glycolipids and glycoproteins.

The bacteria may also or alternatively comprise bacterial metabolites. In this specification the term ‘bacterial metabolites’ includes all molecules produced or modified by the (probiotic) bacteria as a result of bacterial metabolism during growth, survival, persistence, transit or existence of bacteria during probiotic product manufacture and storage and during gastrointestinal transit in a mammal. Examples include all organic acids, inorganic acids, bases, proteins and peptides, enzymes and co-enzymes, amino acids and nucleic acids, carbohydrates, lipids, glycoproteins, lipoproteins, glycolipids, vitamins, all bioactive compounds, metabolites containing an inorganic component, and all small molecules, for example nitrous molecules or molecules containing a sulphurous acid.

Preferably the bacteria comprise whole bacteria, more preferably whole viable bacteria.

Preferably the lactic acid bacterium and/or Bifidobacterium to be used in the present invention is a lactic acid bacterium and/or Bifidobacterium which is generally recognised as safe and, which is preferably GRAS approved.

A skilled person will readily be aware of specific species and or strains of lactic acid bacteria and/or Bifidobacteria from within the genera described herein which are used in the food and/or agricultural industries and which are generally considered suitable for human and/or animal consumption.

Preferably, the lactic acid bacterium and/or Bifidobacterium used in accordance with the present invention is one which is suitable for human and/or animal consumption.

In the present invention, the bacteria used may be of the same type (genus, species and strain) or may comprise a mixture of genera, species and/or strains.

Suitable lactic acid bacteria may be selected from the genera Lactococcus, Lactobacillus, Leuconostoc, Camobacterium, Enterococcus, Propionibacterium, Pediococcus, and Streptococcus and mixtures thereof. Typically, the lactic acid bacteria are selected from the species Leuconostoc spp., Lactococcus cremoris, Lactococcus lactis, Lactobacillus acidophilus, Lactobacillus casei, Lactobacillus kefiri, Lactobacillus bifidus, Lactobacillus brevis, Lactobacillus helveticus, Lactobacillus paracasei, Lactobacillus rhamnosus, Lactobacillus salivarius, Lactobacillus curvatus, Lactobacillus bulgaricus, Lactobacillus sakei, Lactobacillus reuteri, Lactobacillus fermentum, Lactobacillus farciminis, Lactobacillus lactis, Lactobacillus delbreuckii, Lactobacillus plantarum, Lactobacillus paraplantarum, Lactobacillus crispatus, Lactobacillus gasseri, Lactobacillus johnsonii and Lactobacillus jensenii, and combinations of any thereof.

Suitable Bifidobacteria are selected from the species Bifidobacterium lactis, Bifidobacterium bifidium, Bifidobacterium longum, Bifidobacterium animalis, Bifidobacterium breve, Bifidobacterium infantis, Bifidobacterium catenulatum, Bifidobacterium pseudocatenulatum, Bifidobacterium adolescentis, and Bifidobacterium angulatum, and combinations of any thereof.

Preferably, the bacteria used in the present invention are selected from the genera Lactobacillus or Bifidobacterium and mixtures thereof. More preferably, the bacteria used in the present invention are selected from the species Lactobacillus acidophilus, Lactobacillus plantarum, Bifidobacterium animalis, Bifidobacterium lactis, or Bifidobacterium bifidium, and mixtures thereof.

In a particularly preferred embodiment, the bacteria used in the present invention are Lactobacillus acidophilus strain NCFM. Lactobacillus acidophilus NCFM was deposited by Rhodia Chimie, France, at the American Type Culture Collection as PTA-4797 on 15 Nov. 2002.

In one embodiment, the bacterium used in the present invention is a probiotic bacterium. In this specification the term ‘probiotic bacterium’ is defined as covering any non-pathogenic bacterium which, when administered live in adequate amounts, confer a health benefit on the host. These probiotic strains generally have the ability to survive the passage through the upper part of the digestive tract. They are non-pathogenic, non-toxic and exercise their beneficial effect on health on the one hand via ecological interactions with the resident flora in the digestive tract, and on the other hand via their ability to influence the immune system in a positive manner via the “GALT” (gut-associated lymphoid tissue). Depending on the definition of probiotics, these bacteria, when given in a sufficient number, have the ability to progress live through the intestine, however they do not cross the intestinal barrier and their primary effects are therefore induced in the lumen and/or the wall of the gastrointestinal tract. They then form part of the resident flora during the administration period. This colonization (or transient colonization) allows the probiotic bacteria to exercise a beneficial effect, such as the repression of potentially pathogenic micro-organisms present in the flora and interactions with the immune system of the intestine.

In preferred embodiments, the bacterium used in the present invention is a probiotic lactic acid bacterium and/or a probiotic Bifidobacterium.

### Dosage

The lactic acid bacterium and/or Bifidobacterium used in accordance with the present invention (such as a strain of Lactobacillus spp.; for example a strain of Lactobacillus acidophilus and/or Lactobacillus plantarum, and/or a strain of Bifidobacterium spp., for example B. lactis or B. animalis), may comprise from 10⁶ to 10¹² CFU of bacteria/g of support, and more particularly from 10⁸ to 10¹² CFU of bacteria/g of support, preferably 10⁹ to 10¹² CFU/g for the lyophilized form.

Suitably the lactic acid bacterium and/or Bifidobacterium used, in accordance with the present invention (such as a strain of Lactobacillus spp.; for example a strain of Lactobacillus acidophilus and/or Lactobacillus plantarum and/or a strain of Bifidobacterium spp., for example B. lactis or B. animalis), may be administered at a dosage of from about 10⁶ to about 10¹² CFU of microorganism/dose, preferably about 10⁸ to about 10¹² CFU of microorganism/dose. By the term “per dose” it is meant that this amount of microorganism is provided to a subject either per day or per intake, preferably per day. For example, if the microorganism is to be administered in a food product (for example in a yoghurt)—then the yoghurt will preferably contain from about 10⁸ to 10¹² CFU of the microorganism. Alternatively, however, this amount of microorganism may be split into multiple administrations each consisting of a smaller amount of microbial loading—so long as the overall amount of microorganism received by the subject in any specific time (for instance each 24 hour period) is from about 10⁶ to about 10¹² CFU of microorganism, preferably 10⁸ to about 10¹² CFU of microorganism.

In accordance with the present invention an effective amount of at least one strain of a microorganism may be at least 10⁶ CFU of microorganism/dose, preferably from about 10⁶ to about 10¹² CFU of microorganism/dose, preferably about 10⁸ to about 10¹² CFU of microorganism/dose.

In one embodiment, preferably the lactic acid bacterium and/or Bifidobacterium used in accordance with the present invention (such as a strain of Lactobacillus spp.; for example a strain of Lactobacillus acidophilus, and/or Lactobacillus plantarum and/or a strain of Bifidobacterium spp., for example B. lactis or B. animalis) may be administered at a dosage of from about 10⁶ to about 10¹² CFU of microorganism/day, preferably about 10⁸ to about 10¹² CFU of microorganism/day. Hence, the effective amount in this embodiment may be from about 10⁶ to about 10¹² CFU of microorganism/day, preferably about 10⁸ to about 10¹² CFU of microorganism/day.

CFU stands for “colony-forming units”. By ‘support’ is meant the food product, dietary supplement or the pharmaceutically acceptable support.

### Subjects/Medical Indications

The lactic acid bacteria and/or Bifidobacteria to which the present invention relates are administered to a mammal, including for example livestock (including cattle, horses, pigs, chickens and sheep), and humans. In some aspects of the present invention the mammal is a companion animal (including pets), such as a dog or a cat for instance. In some aspects of the present invention, the subject may suitably be a human.

The present inventors have surprisingly found that lactic acid bacteria and/or Bifidobacteria to which the present invention relates are capable of lowering adipose tissue inflammation in mammals. There is epidemiological evidence in the literature showing a statistical relationship between inflammation, obesity and insulin resistance in humans (Cani et al., Diabetes, 2007, 56, 1761-1772, and references cited therein). This finding therefore confers the potential for the lactic acid bacteria and/or Bifidobacteria to be useful in the treatment of obesity, diabetes and related conditions, metabolic diseases and cardiovascular consequences in mammals.

According to Berg and Scherer, Circulation Research, 2005, 96, 939, recent evidence highlights the role of adipose tissue in the development of a systemic inflammatory state that contributes to obesity-associated vasculopathy and cardiovascular risk. Circulating mediators of inflammation participate in the mechanisms of vascular insult and atheromatous change, and many of these inflammatory proteins are secreted directly from adipocytes and adipose tissue-derived macrophages. Several factors linking obesity with an increased cardiovascular risk have been identified. The adipocyte-specific secretory protein adiponectin is a particularly promising candidate in this context. Its levels are decreased in obesity.

The targeted suppression of various proinflammatory cascades in adipocytes specifically represents a new therapeutic opportunity for the cardiovascular disease area. Suppression of adipose tissue inflammation would therefore be expected to provide a therapeutic benefit in the treatment of cardiovascular diseases.

Examples of cardiovascular diseases potentially treatable according to the lactic acid bacteria and/or Bifidobacteria used in the present invention include aneurysm, angina, atherosclerosis, cerebrovascular accident (stroke), cerebrovascular disease, congestive heart failure (CHF), coronary artery disease, myocardial infarction (heart attack) and peripheral vascular disease.

An aneurysm is a localized, blood-filled dilation (balloon-like bulge) of a blood vessel caused by disease or weakening of the vessel wall. Aneurysms most commonly occur in arteries at the base of the brain (the circle of Willis) and in the aorta (the main artery coming out of the heart, a so-called aortic aneurysm). As the size of an aneurysm increases, there is an increased risk of rupture, which can result in severe hemorrhage or other complications including sudden death.

Angina pectoris, commonly known as angina, is severe chest pain due to ischemia (a lack of blood and hence oxygen supply) of the heart muscle, generally due to obstruction or spasm of the coronary arteries (the heart's blood vessels). Coronary artery disease, the main cause of angina, is due to atherosclerosis of the cardiac arteries.

Atherosclerosis is the condition in which an artery wall thickens as the result of a build up of fatty materials such as cholesterol. It is a syndrome affecting arterial blood vessels. It is a chronic inflammatory response in the walls of arteries, in large part due to the accumulation of macrophage white blood cells and promoted by low density (especially small particle) lipoproteins (plasma proteins that carry cholesterol and triglycerides) without adequate removal of fats and cholesterol from the macrophages by functional high density lipoproteins (HDL). It is commonly referred to as a hardening or furring of the arteries. It is caused by the formation of multiple plaques within the arteries.

A stroke is the rapidly developing loss of brain function(s) due to disturbance in the blood supply to the brain. This can be due to ischemia (lack of blood supply) caused by thrombosis or embolism or due to a hemorrhage. As a result, the affected area of the brain is unable to function, leading to inability to move one or more limbs on one side of the body, inability to understand or formulate speech, or see one side of the visual field, and ultimately to death.

Cerebrovascular disease is a group of brain dysfunctions related to disease of blood vessels supplying the brain. Hypertension is the most important cause that damages the blood vessel lining endothelium exposing the underlying collagen where platelets aggregate to initiate a repairing process which is not always complete and perfect. Sustained hypertension permanently changes the architecture of the blood vessels making them narrow, stiff, deformed and uneven which are more vulnerable to fluctuations of blood pressure. A fall in blood pressure during sleep can lead to marked reduction in blood flow in the narrowed blood vessels causing ischemic stroke in the morning whereas a sudden rise in blood pressure can cause tearing of the blood vessels causing intracranial hemorrhage during excitation at daytime. Primarily people who are elderly, diabetic, smoker, or have ischemic heart disease, have cerebrovascular disease. All diseases related to artery dysfunction can be classified under a disease as known as macrovascular disease. This is a simplistic study by which arteries are blocked by fatty deposits or by a blood clot. The results of cerebrovascular disease can include a stroke, or even sometimes a hemorrhagic stroke. Ischemia or other blood vessel dysfunctions can affect one during a cerebrovascular accident.

Heart failure is a global term for the physiological state in which cardiac output is insufficient for the body's needs. This may occur when the cardiac output is low (often termed “congestive heart failure”). Common causes of heart failure include myocardial infarction and other forms of ischemic heart disease, hypertension, valvular heart disease and cardiomyopathy.

Coronary disease (or coronary heart disease) refers to the failure of coronary circulation to supply adequate circulation to cardiac muscle and surrounding tissue. It is most commonly equated with atherosclerotic coronary artery disease, but coronary disease can be due to other causes, such as coronary vasospasm. It is possible for the stenosis to be caused by the spasm.

Myocardial infarction, commonly known as a heart attack, occurs when the blood supply to part of the heart is interrupted causing some heart cells to die. This is most commonly due to occlusion (blockage) of a coronary artery following the rupture of a vulnerable atherosclerotic plaque, which is an unstable collection of lipids (like cholesterol) and white blood cells (especially macrophages) in the wall of an artery. The resulting ischemia (restriction in blood supply) and oxygen shortage, if left untreated for a sufficient period of time, can cause damage and/or death (infarction) of heart muscle tissue (myocardium).

Peripheral vascular disease (PVD), also known as peripheral artery disease (PAD) or peripheral artery occlusive disease (PAOD), includes all diseases caused by the obstruction of large arteries in the arms and legs. PVD can result from atherosclerosis, inflammatory processes leading to stenosis, an embolism or thrombus formation. It causes either acute or chronic ischemia (lack of blood supply), typically of the legs.

In further embodiments, lactic acid bacteria may be used according to the present invention to lower tissue inflammation (particularly, although not exclusively, liver tissue inflammation, muscle tissue inflammation and/or adipose tissue inflammation) in a mammal.

In one embodiment, lactic acid bacteria may be used according to the present invention to lower liver tissue inflammation. This confers the potential for the application of the lactic acid bacteria in the treatment of hepatitis, which is characterised by the destruction of a number of liver cells and the presence of inflammatory cells in the liver tissue.

Hepatitis can be divided into two subgroups according to its duration: acute hepatitis (lasting less than six months) and chronic hepatitis (lasting longer than six months). Hepatitis may be also classified according to its cause: for example, hepatitis may comprise Infectious viral hepatitis (such as hepatitis A, hepatitis B, hepatitis C, hepatitis D and hepatitis E), hepatitis caused by other viral diseases (such as mononucleosis and cytomegalovirus), hepatitis caused by severe bacterial infections or amoebic infections, hepatitis caused by medicines, hepatitis caused by toxins such as alcohol, autoimmune hepatitis (in which a number of liver cells are destroyed by the patient's own immune system) and hepatitis caused by congenital metabolic disorders, such as Wilson's disease (disorder of the body's copper metabolism) and haemochromatosis (disorder of the body's iron metabolism).

In one embodiment, lactic acid bacteria may be used according to the present invention to lower muscle tissue inflammation. This confers the potential for the application of the bacteria in the treatment of myositis, in which the muscle fibers and skin are inflamed and damaged, resulting in muscle weakness.

There are several types of myositis that affect different parts of the body. Particular forms of myositis treatable according to the present invention include: polymyositis (PM) (in which muscles in many parts of the body, and especially those parts closest to the trunk, are inflamed); dermatomyositis (DM) (which affects both the muscle fibers and skin by damaging capillaries that supply blood to the muscle and skin), inclusion body myositis (IBM) which is characterized by gradual weakening of muscles throughout the body, including the wrists or fingers, development of dysphagia, and atrophy of forearms and/or thigh muscles; and juvenile myositis (JM), which involves muscle weakness, skin rash, and dysphagia in children.

The lactic acid bacteria and/or Bifidobacteria to which the present invention relates are suitable for administration to both diabetic and obese mammals. They could also be suitable for diabetic and non-obese mammals, as well as to obese mammals possessing the risk factors for diabetes, but not yet in a diabetic state. This aspect is discussed in more detail below.

In preferred embodiments, the condition being treated or prevented is diet-induced and/or diet-associated. The present inventors have surprisingly found that the lactic acid bacteria and/or Bifidobacteria can be used in accordance with the present invention to treat a number of diet-induced and/or diet-associated conditions, as described in more detail below.

The use of lactic acid bacteria and/or Bifidobacteria according to the present invention is particularly advantageous for mammals unable to alter their high-fat diet and/or unable to lose weight, but nevertheless requiring improvement of some metabolic parameters to avoid further complications of their diabetes and/or obesity.

The compositions of the present invention, as defined below, may additionally be suitable for treating a number of conditions in diabetic and/or obese persons. Without wishing to be bound by theory, it is believed that the compositions are effective without any concomitant decrease in weight gain. In this specification, by ‘without a concomitant decrease in weight gain’ is meant, over the course of the treatment, the weight of the treated subject decreases to a lesser extent compared with known treatments of diabetes and obesity (which are generally aimed at treating both conditions together). Examples of known treatments of diabetes include diet, exercise, insulin therapy (such as those described and exemplified above) and oral antidiabetic drugs and insulin analogs (such as those described and exemplified above).

In one embodiment, the term ‘without a concomitant decrease in weight gain’ means subjects whose weight increases over the course of the treatment, compared with the subject's weight at the commencement of the treatment.

In another embodiment, the term ‘without a concomitant decrease in weight gain’ means subjects whose weight remains substantially unchanged (preferably unchanged) over the course of the treatment, compared with the subject's weight at the commencement of the treatment. In this regard, the term ‘substantially unchanged’ means the weight does not change to a significant extent (suitably, by less than 5%; preferably, by less than 2%; more preferably, by less than 1%; even more preferably, by less than 0.5%; most preferably, by less than 0.25%) over the course of the treatment.

In a further embodiment, the term ‘without a concomitant decrease in weight gain’ means subjects whose weight decreases over the course of the treatment, compared with the subject's weight at the commencement of the treatment, but to a lesser extent compared with known treatments of diabetes and obesity.

Preferably, the term ‘without a concomitant decrease in weight gain’ comprises subjects whose weight increases or remains substantially unchanged (as defined and exemplified above; preferably unchanged) over the course of the treatment, compared with the subject's weight at the commencement of the treatment.

In particular, the use of lactic acid bacteria and/or Bifidobacteria according to the present invention is advantageous for those diabetic and/or obese patients whom (especially, although not exclusively, for reasons unconnected to their conditions) continue to gain weight over the course of the treatment, as treatment with the lactic acid bacteria and/or Bifidobacteria may offset the metabolic conditions of this continued weight gain.

In particular, the use of lactic acid bacteria and/or Bifidobacteria according to the present invention is suitable for the treatment of mammals ingesting a high-fat diet. This aspect is discussed in more detail below.

The present inventors have surprisingly found that treatment with lactic acid bacteria and/or Bifidobacteria according to the present invention decreases blood glucose levels in mammals concomitantly gaining weight. The finding that decrease of glucose level is not the consequence of a weight loss is contrary to what would have been expected, as the prior art documents generally describe the use of lactic acid bacteria, including probiotics, to treat and/or prevent both obesity and diabetes concomitantly.

The compositions are suitable for use in obese and diabetic patients. In this specification the term ‘diabetes’ includes all forms of diabetes which, as noted above, is characterised by disordered metabolism and abnormally high blood sugar (hyperglycaemia) resulting from insufficient levels of the hormone insulin. The term therefore includes Type 1 diabetes, Type 2 diabetes, gestational diabetes, and impaired glucose tolerance. Type 1 diabetes is characterised by loss of the insulin-producing beta cells of the islets of Langerhans in the pancreas, leading to a deficiency of insulin. Type 2 diabetes mellitus is characterised by insulin resistance or reduced insulin sensitivity, combined with reduced insulin secretion. Gestational diabetes is formally defined as “any degree of glucose intolerance with onset or first recognition during pregnancy”. Impaired Glucose Tolerance (IGT) is a pre-diabetic state of dysglycemia that is associated with insulin resistance and increased risk of cardiovascular pathology. According to the criteria of the World Health Organization and the American Diabetes Association, impaired glucose tolerance is defined as two-hour glucose levels of 140 to 199 mg per dL (7.8 to 11.0 mmol) on the 75-g oral glucose tolerance test. A patient is said to be under the condition of IGT when he/she has an intermediately raised glucose level after 2 hours, but less than would qualify for type 2 diabetes mellitus. The fasting glucose may be either normal or mildly elevated. IGT may precede type 2 diabetes mellitus by many years. IGT is also a risk factor for mortality.

In this specification, the term obesity is linked to body mass index (BMI). The body mass index (BMI) (calculated as weight in kilograms divided by the square of height in metres) is the most commonly accepted measurement for overweight and/or obesity. A BMI exceeding 25 is considered overweight. Obesity is defined as a BMI of 30 or more, with a BMI of 35 or more considered as serious comorbidity obesity and a BMI of 40 or more considered morbid obesity.

As noted above, the term “obesity” as used herein includes obesity, comorbidity obesity and morbid obesity. Therefore, the term “obese” as used here may be defined as a subject having a BMI of more than or equal to 30. In some embodiments, suitably an obese subject may have a BMI of more than or equal to 30, suitably 35, suitably 40.

While the composition of the invention is particularly suitable for use in patients who are both diabetic and obese, the composition is also suitable for those who are diabetic but not obese. It may also be suitable for use in obese patients possessing the risk factors for diabetes, but not yet in a diabetic state, as it could be expected that an obese person (but not diabetic), could limit the metabolic consequences of his obesity, i.e. the diabetes or at least insulino-resistance development.

In one embodiment, lactic acid bacteria and/or Bifidobacteria, especially probiotic bacteria such as Lactobacillus acidophilus and/or Bifidobacterium animalis, may be used for treating diabetes, particularly although not exclusively the metabolic complications of diabetes, in a mammal. In preferred embodiments, the diabetes is diet-induced and/or diet-associated.

In one embodiment, lactic acid bacteria and/or Bifidobacteria, especially probiotic bacteria such as Lactobacillus acidophilus and/or Bifidobacterium animalis, may be used for treating obesity, particularly although not exclusively the metabolic complications of obesity, in a mammal. In preferred embodiments, the obesity is diet-induced and/or diet-associated.

In particular, lactic acid bacteria and/or Bifidobacteria, especially probiotic bacteria such as Lactobacillus acidophilus and/or Bifidobacterium animalis, have been shown to improve the glycaemic parameters in diabetes and/or obese mammals. Furthermore, microorganisms, especially probiotic bacteria such as Lactobacillus acidophilus and/or Bifidobacterium animalis, can be used to reduce insulin resistance in diabetes and/or obese mammals.

It has been surprisingly found that some or all of the above effects can take place without a concomitant decrease in weight gain (as defined and exemplified above). The lactic acid bacteria and/or Bifidobacteria, especially probiotic bacteria such as Lactobacillus acidophilus and/or Bifidobacterium animalis, may be particularly useful for limiting diabetes (particularly although not exclusively the metabolic complications of diabetes) in subjects unable or unwilling to alter their diet, exercise or otherwise lose weight.

In another embodiment, lactic acid bacteria and/or Bifidobacteria, especially probiotic bacteria such as Lactobacillus acidophilus and/or Bifidobacterium animalis, may be used for treating metabolic syndrome in a mammal. Metabolic syndrome is a combination of medical disorders that increase the risk of developing cardiovascular disease and diabetes. Metabolic syndrome is also known as metabolic syndrome X, syndrome X, insulin resistance syndrome, Reaven's syndrome or CHAOS (Australia).

The invention therefore comprises in an additional aspect use of a bacterium selected from a lactic acid bacterium, a Bifidobacterium or a mixture of any thereof in the manufacture of a food product, dietary supplement or medicament for treating metabolic syndrome in a mammal.

There is currently no single accepted definition of metabolic syndrome. The World Health Organization criteria (1999) require presence of diabetes mellitus, impaired glucose tolerance, impaired fasting glucose or insulin resistance, AND two of the following:
blood pressure: ≧140/90 mmHg
dyslipidaemia: triglycerides (TG): ≧1.695 mmol/L and high-density lipoprotein cholesterol (HDL-C)≦0.9 mmol/L (male), ≦1.0 mmol/L (female)
central obesity: waist:hip ratio >0.90 (male); >0.85 (female), and/or body mass index >30 kg/m²
microalbuminuria: urinary albumin excretion ratio ≧20 mg/min or albumin:creatinine ratio ≧30 mg/g.

The European Group for the Study of Insulin Resistance (1999) requires insulin resistance defined as the top 25% of the fasting insulin values among non-diabetic individuals AND two or more of the following:
central obesity: waist circumference ≧94 cm (male), ≧80 cm (female)
dyslipidaemia: TG ≧2.0 mmol/L and/or HDL-C <1.0 mg/dL or treated for dyslipidaemia
hypertension: blood pressure ≧140/90 mmHg or antihypertensive medication
fasting plasma glucose ≧6.1 mmol/L

The US National Cholesterol Education Program (NCEP) Adult Treatment Panel III (2001) requires at least three of the following:
central obesity: waist circumference ≧102 cm or 40 inches (male), ≧88 cm or 36 inches (female)
dyslipidaemia: TG ≧1.695 mmol/L (150 mg/dl)
dyslipidaemia: HDL-C <40 mg/dL (male), <50 mg/dL (female)
blood pressure ≧130/85 mmHg
fasting plasma glucose ≧6.1 mmol/L (110 mg/dl)

The lactic acid bacteria and/or Bifidobacteria used in the present invention could also have the advantage to decrease intestinal permeability. Without wishing to be bound by theory, it is possible that modification of intestinal permeability may allow the lactic acid bacteria and/or Bifidobacteria, especially probiotic bacteria such as Lactobacillus acidophilus and/or Bifidobacterium animalis, to be effective in the treatment of conditions such as diabetes and metabolic syndrome, and complications and consequences thereof.

The invention therefore comprises in a further aspect use of a bacterium selected from a lactic acid bacterium, a Bifidobacterium or a mixture of any thereof in the manufacture of a food product, dietary supplement or medicament for decreasing intestinal permeability in a diabetic and/or obese mammal.

In one embodiment, lactic acid bacteria and/or Bifidobacteria, especially probiotic bacteria such as Lactobacillus acidophilus and/or Bifidobacterium animalis, could be used for decreasing the risk of infection in a diabetic and/or obese mammal. Without wishing to be bound by theory, it is believed that as the bacteria decrease the permeability of the intestinal cells, the passage of pathogenic bacteria from the gut lumen to the blood circulation, and therefrom to organs, could be decreased. In particular, lactic acid bacteria, especially probiotic bacteria, may be used for decreasing the risk of foot infection, which is common in diabetic patients.

The invention therefore comprises in an additional aspect use of a bacterium selected from a lactic acid bacterium, a Bifidobacterium and a mixture of any thereof in the manufacture of a food product, dietary supplement or medicament for decreasing the risk of infection in a diabetic and/or obese mammal.

In this specification the term “treatment” or “treating” refers to any administration of the lactic acid bacteria and/or Bifidobacteria according to the present invention and includes: (1) preventing the specified disease from occurring in an animal which may be predisposed to the disease but does not yet experience or display the pathology or symptomatology of the disease (including prevention of one or more risk factors associated with the disease); (2) inhibiting the disease in an animal that is experiencing or displaying the pathology or symptomatology of the diseased (i.e., arresting further development of the pathology and/or symptomatology), or (3) ameliorating the disease in an animal that is experiencing or displaying the pathology or symptomatology of the diseased (i.e., reversing the pathology and/or symptomatology).

### Diet

As noted above, diabetic and/or obese mammals treated with bacteria according to the present invention may continue to ingest a high-fat diet while mitigating the metabolic consequences of their condition(s). In this specification the term ‘high-fat diet’ means a diet generally containing at least 20%, preferably at least 25%, such as at least 30%, for example at least 35%, such as at least 40%, for example at least 45%, such as at least 50%, for example at least 55%, such as at least 60%, for example at least 65%, such as at least 70%, for example at least 75%, such as at least 80%, for example at least 85%, such as at least 90% of calories from fat.

In some embodiments, diabetic and/or obese mammals treated with bacteria according to the present invention may ingest a high-carbohydrate diet while mitigating the metabolic consequences of their condition(s). In this specification the term ‘high-fat diet’ means a diet generally containing at least 50%, for example at least 55%, such as at least 60%, for example at least 65%, such as at least 70%, for example at least 75%, such as at least 80%, for example at least 85%, such as at least 90% of calories from carbohydrate.

### Compositions

While is it possible to administer lactic acid bacteria and/or Bifidobacteria alone according to the present invention, the lactic acid bacteria and/or Bifidobacteria are typically and preferably administered on or in a support as part of a product, in particular as a component of a food product, a dietary supplement or a pharmaceutical formulation. These products typically contain additional components well known to those skilled in the art.

Any product which can benefit from the composition may be used in the present invention. These include but are not limited to foods, particularly fruit conserves and dairy foods and dairy food-derived products, and pharmaceutical products. The lactic acid bacteria may be referred to herein as “the composition of the present invention” or “the composition”.

### Food

In one embodiment, the lactic acid bacteria and/or Bifidobacteria are employed according to the invention in a food product such as a food supplement, a drink or a powder based on milk. Here, the term “food” is used in a broad sense—and covers food for humans as well as food for animals (i.e. a feed). In a preferred aspect, the food is for human consumption.

The food may be in the form of a solution or as a solid—depending on the use and/or the mode of application and/or the mode of administration.

When used as, or in the preparation of, a food, such as functional food, the composition of the present invention may be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the composition of the present invention can be used as an ingredient to soft drinks, a fruit juice or a beverage comprising whey protein, health teas, cocoa drinks, milk drinks and lactic acid bacteria drinks, yoghurt and drinking yoghurt, cheese, ice cream, water ices and desserts, confectionery, biscuits cakes and cake mixes, snack foods, balanced foods and drinks, fruit fillings, care glaze, chocolate bakery filling, cheese cake flavoured filling, fruit flavoured cake filling, cake and doughnut icing, instant bakery filling creams, fillings for cookies, ready-to-use bakery filling, reduced calorie filling, adult nutritional beverage, acidified soy/juice beverage, aseptic/retorted chocolate drink, bar mixes, beverage powders, calcium fortified soy/plain and chocolate milk, calcium fortified coffee beverage.

The composition can further be used as an ingredient in food products such as American cheese sauce, anti-caking agent for grated & shredded cheese, chip dip, cream cheese, dry blended whip topping fat free sour cream, freeze/thaw dairy whipping cream, freeze/thaw stable whipped tipping, low fat and light natural cheddar cheese, low fat Swiss style yoghurt, aerated frozen desserts, hard pack ice cream, label friendly, improved economics & indulgence of hard pack ice cream, low fat ice cream: soft serve, barbecue sauce, cheese dip sauce, cottage cheese dressing, dry mix Alfredo sauce, mix cheese sauce, dry mix tomato sauce and others.

The term “dairy product” as used herein is meant to include a medium comprising milk of animal and/or vegetable origin. As milk of animal origin there can be mentioned cow's, sheep's, goat's or buffalo's milk. As milk of vegetable origin there can be mentioned any fermentable substance of vegetable origin which can be used according to the invention, in particular originating from soybeans, rice or cereals.

Still more preferably the food product employed according to the invention is a fermented milk or humanized milk.

For certain aspects, preferably the present invention may be used in connection with yoghurt production, such as fermented yoghurt drink, yoghurt, drinking yoghurt, cheese, fermented cream, milk based desserts and others.

Suitably, the composition can be further used as an ingredient in one or more of cheese applications, meat applications, or applications comprising protective cultures.

The present invention also provides a method of preparing a food or a food ingredient, the method comprising admixing the composition according to the present invention with another food ingredient.

Advantageously, the present invention relates to products that have been contacted with the composition of the present invention (and optionally with other components/ingredients), wherein the composition is used in an amount to be capable of improving the nutrition and/or health benefits of the product.

As used herein the term “contacted” refers to the indirect or direct application of the composition of the present invention to the product. Examples of the application methods which may be used, include, but are not limited to, treating the product in a material comprising the composition, direct application by mixing the composition with the product, spraying the composition onto the product surface or dipping the product into a preparation of the composition.

Where the product of the invention is a foodstuff, the composition of the present invention is preferably admixed with the product. Alternatively, the composition may be included in the emulsion or raw ingredients of a foodstuff. In a further alternative, the composition may be applied as a seasoning, glaze, colorant mixture, and the like.

For some applications, it is important that the composition is made available on or to the surface of a product to be affected/treated. This allows the composition to impart one or more of the following favourable characteristics: nutrition and/or health benefits.

The compositions of the present invention may be applied to intersperse, coat and/or impregnate a product with a controlled amount of a viable microorganism.

Preferably, the composition is used to ferment milk or sucrose fortified milk or lactic media with sucrose and/or maltose where the resulting media containing all components of the composition—i.e. said microorganism according to the present invention—can be added as an ingredient to yoghurt milk in suitable concentrations—such as for example in concentrations in the final product which offer a daily dose of 10⁶-10¹⁰ cfu. The microorganism according to the present invention may be used before or after fermentation of the yoghurt.

For some aspects the microorganisms according to the present invention are used as, or in the preparation of, animal feeds, such as livestock feeds, in particular poultry (such as chicken) feed, or pet food.

Advantageously, where the product is a food product, the lactic acid bacteria should remain effective through the normal “sell-by” or “expiration” date during which the food product is offered for sale by the retailer. Preferably, the effective time should extend past such dates until the end of the normal freshness period when food spoilage becomes apparent. The desired lengths of time and normal shelf life will vary from foodstuff to foodstuff and those of ordinary skill in the art will recognise that shelf-life times will vary upon the type of foodstuff, the size of the foodstuff, storage temperatures, processing conditions, packaging material and packaging equipment.

### Food Ingredient

The composition of the present invention may be used as a food ingredient and/or feed ingredient.

As used herein the term “food ingredient” or “feed ingredient” includes a formulation which is or can be added to functional foods or foodstuffs as a nutritional supplement.

The food ingredient may be in the form of a solution or as a solid—depending on the use and/or the mode of application and/or the mode of administration.

### Food Supplements

The composition of the present invention may be—or may be added to—food supplements (also referred to herein as dietary supplements).

### Functional Foods

The composition of the present invention may be—or may be added to—functional foods.

As used herein, the term “functional food” means food which is capable of providing not only a nutritional effect, but is also capable of delivering a further beneficial effect to consumer.

Accordingly, functional foods are ordinary foods that have components or ingredients (such as those described herein) incorporated into them that impart to the food a specific functional—e.g. medical or physiological benefit—other than a purely nutritional effect.

Although there is no legal definition of a functional food, most of the parties with an interest in this area agree that they are foods marketed as having specific health effects beyond basic nutritional effects.

Some functional foods are nutraceuticals. Here, the term “nutraceutical” means a food which is capable of providing not only a nutritional effect and/or a taste satisfaction, but is also capable of delivering a therapeutic (or other beneficial) effect to the consumer. Nutraceuticals cross the traditional dividing lines between foods and medicine.

### Medicament

The term “medicament” as used herein encompasses medicaments for both human and animal usage in human and veterinary medicine. In addition, the term “medicament” as used herein means any substance which provides a therapeutic and/or beneficial effect. The term “medicament” as used herein is not necessarily limited to substances which need Marketing Approval, but may include substances which can be used in cosmetics, nutraceuticals, food (including feeds and beverages for example), probiotic cultures, and natural remedies. In addition, the term “medicament” as used herein encompasses a product designed for incorporation in animal feed, for example livestock feed and/or pet food.

### Pharmaceutical

The composition of the present invention may be used as—or in the preparation of—a pharmaceutical. Here, the term “pharmaceutical” is used in a broad sense—and covers pharmaceuticals for humans as well as pharmaceuticals for animals (i.e. veterinary applications). In a preferred aspect, the pharmaceutical is for human use and/or for animal husbandry.

The pharmaceutical can be for therapeutic purposes—which may be curative or palliative or preventative in nature. The pharmaceutical may even be for diagnostic purposes.

A pharmaceutically acceptable support may be for example a support in the form of compressed tablets, tablets, capsules, ointments, suppositories or drinkable solutions. Other suitable forms are provided below.

When used as—or in the preparation of—a pharmaceutical, the composition of the present invention may be used in conjunction with one or more of: a pharmaceutically acceptable carrier, a pharmaceutically acceptable diluent, a pharmaceutically acceptable excipient, a pharmaceutically acceptable adjuvant, a pharmaceutically active ingredient.

The pharmaceutical may be in the form of a solution or as a solid—depending on the use and/or the mode of application and/or the mode of administration.

The lactic acid bacteria of the present invention may be used as pharmaceutical ingredients. Here, the composition may be the sole active component or it may be at least one of a number (i.e. 2 or more) of active components.

The pharmaceutical ingredient may be in the form of a solution or as a solid—depending on the use and/or the mode of application and/or the mode of administration.

The lactic acid bacteria may be used according to the present invention in any suitable form—whether when alone or when present in a combination with other components or ingredients. The lactic acid bacteria used in the present invention may be referred to herein as “the composition”. Likewise, combinations comprising the composition of the present invention and other components and/or ingredients (i.e. ingredients—such as food ingredients, functional food ingredients or pharmaceutical ingredients) may be used in any suitable form.

The lactic acid bacteria of the present invention may be used in the form of solid or liquid preparations or alternatives thereof. Examples of solid preparations include, but are not limited to tablets, capsules, dusts, granules and powders which may be wettable, spray-dried or freeze-dried. Examples of liquid preparations include, but are not limited to, aqueous, organic or aqueous-organic solutions, suspensions and emulsions.

Suitable examples of forms include one or more of: tablets, pills, capsules, ovules, solutions or suspensions, which may contain flavouring or colouring agents, for immediate-, delayed-, modified-, sustained-, pulsed- or controlled-release applications.

By way of example, if the composition of the present invention is used in a tablet form—such for use as a functional ingredient—the tablets may also contain one or more of: excipients such as microcrystalline cellulose, lactose, sodium citrate, calcium carbonate, dibasic calcium phosphate and glycine; disintegrants such as starch (preferably corn, potato or tapioca starch), sodium starch glycollate, croscarmellose sodium and certain complex silicates; granulation binders such as polyvinylpyrrolidone, hydroxypropylmethylcellulose (HPMC), hydroxypropylcellulose (HPC), sucrose, gelatin and acacia; lubricating agents such as magnesium stearate, stearic acid, glyceryl behenate and talc may be included.

Examples of nutritionally acceptable carriers for use in preparing the forms include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethylcellulose, polyvinylpyrrolidone, and the like.

Preferred excipients for the forms include lactose, starch, a cellulose, milk sugar or high molecular weight polyethylene glycols.

For aqueous suspensions and/or elixirs, the composition of the present invention may be combined with various sweetening or flavouring agents, colouring matter or dyes, with emulsifying and/or suspending agents and with diluents such as water, propylene glycol and glycerin, and combinations thereof.

The forms may also include gelatin capsules; fibre capsules, fibre tablets etc.; or even fibre beverages.

Further examples of form include creams. For some aspects the microorganism used in the present invention may be used in pharmaceutical and/or cosmetic creams such as sun creams and/or after-sun creams for example.

In one aspect, the composition according to the present invention may be administered in an aerosol, for example by way of a nasal spray, for instance for administration to the respiratory tract.

The composition of the present invention may additionally contain one or more prebiotics. Prebiotics are a category of functional food, defined as non-digestible food ingredients that beneficially affect the host by selectively stimulating the growth and/or activity of one or a limited number of bacteria (particularly, although not exclusively, probiotics and/or lactic acid bacteria) in the colon, and thus improve host health. Typically, prebiotics are carbohydrates (such as oligosaccharides), but the definition does not preclude non-carbohydrates. The most prevalent forms of prebiotics are nutritionally classed as soluble fibre. To some extent, many forms of dietary fibre exhibit some level of prebiotic effect.

Examples of suitable prebiotics include alginate, xanthan, pectin, locust bean gum (LBG), inulin, guar gum, galacto-oligosaccharide (GOS), fructo-oligosaccharide (FOS), polydextrose (i.e. Litesse®), lactitol, lactosucrose, soybean oligosaccharides, palatinose, isomalto-oligosaccharides, gluco-oligosaccharides and xylo-oligosaccharides.

It is envisaged within the scope of the present invention that the embodiments of the invention can be combined such that combinations of any of the features described herein are included within the scope of the present invention. In particular, it is envisaged within the scope of the present invention that any of the therapeutic effects of the bacteria may be exhibited concomitantly.

Therefore, in a preferred aspect of the invention, there is provided use of a bacterium selected from a lactic acid bacterium, a Bifidobacterium or a mixture of any thereof in the manufacture of a food product, dietary supplement or medicament for reducing the blood glucose level with a concomitant lowering of adipose tissue inflammation in a mammal.

In a further preferred aspect of the invention, there is provided use of a bacterium selected from a lactic acid bacterium, a Bifidobacterium or a mixture of any thereof in the manufacture of a food product, dietary supplement or medicament for reducing insulin resistance with a concomitant lowering of adipose tissue inflammation in a mammal.

In a yet further preferred aspect of the invention, there is provided use of a bacterium selected from a lactic acid bacterium, a Bifidobacterium or a mixture of any thereof in the manufacture of a food product, dietary supplement or medicament for treating diabetes with a concomitant lowering of adipose tissue inflammation in a mammal.

In a still further preferred aspect of the invention, there is provided use of a bacterium selected from a lactic acid bacterium, a Bifidobacterium or a mixture of any thereof in the manufacture of a food product, dietary supplement or medicament for decreasing the metabolic consequences of diabetes with a concomitant lowering of adipose tissue inflammation in a mammal.

In a yet further preferred aspect of the invention, there is provided use of a bacterium selected from a lactic acid bacterium, a Bifidobacterium or a mixture of any thereof in the manufacture of a food product, dietary supplement or medicament for improving glucose tolerance with a concomitant lowering of adipose tissue inflammation in a mammal.

In a still further preferred aspect of the invention, there is provided use of a lactic acid bacterium or a mixture thereof in the manufacture of a food product, dietary supplement or medicament for increasing fed insulin secretion with a concomitant lowering of tissue inflammation (particularly although not exclusively liver tissue inflammation, adipose tissue inflammation or muscle tissue inflammation) in a mammal.

### EXAMPLES

### Example 1

### Evolution of the Glycaemic and Weight Parameters on 2 Sets of Mice Feeding with Lactobacillus acidophilus NCFM

### Material and Methods:

### Animals and Reagents

Six weeks old C57BU6 male mice were obtained from Harlan (Le Genest St Isle, France). Animal experiments were performed in an accredited establishment (A 59-35009) according to Directive 86/609/EEC. Animals were group housed (5/cage) and had free access to the chow and tap water.

Ten mice were fed with the control diet listed in Table 1 below (obtained from Research Diet, New Brunswick, N.J., USA, —formula D12450B). 10 other mice were fed with the high-fat diet (HFD) listed in Table 1 below (obtained from Research Diet, New Brunswick, N.J., USA—formula D12492) in order for them to become “naturally” both obese and diabetic. The first set of mice contains non-diabetic and non-obese mice, and is used as a control. The second set contains diabetic and obese mice.

TABLE 1
High-fat and control diets

| Product | Control diet | | High-fat diet (HFD) | |
|---|---|---|---|---|
| Component | g % | kcal % | g % | kcal % |
| Protein | 19.2 | 20 | 26.2 | 20 |
| Carbohydrate | 67.3 | 70 | 26.3 | 20 |
| Fat | 4.3 | 10 | 34.9 | 60 |
| | Total 3.85 kcal/g | | Total 5.24 kcal/g | |
| Ingredient | g | kcal | g | Kcal |
| Casein, 80 mesh | 200 | 800 | 200 | 800 |
| L-Cystine | 3 | 12 | 3 | 12 |
| Corn Starch | 315 | 1260 | 0 | 0 |
| Maltodextrin 10 | 35 | 140 | 125 | 500 |
| Sucrose | 350 | 1400 | 68.8 | 275.2 |
| Cellulose, BW200 | 50 | 0 | 50 | 0 |
| Soybean Oil | 25 | 225 | 25 | 225 |
| Lard | 20 | 180 | 245 | 2205 |
| Mineral Mix S10026 | 10 | 0 | 10 | 0 |
| Dicalcium Phosphate | 13 | 0 | 13 | 0 |
| Calcium Carbonate | 5.5 | 0 | 5.5 | 0 |
| Potassium Citrate | 16.5 | 0 | 16.5 | 0 |
| monohydrate | | | | |
| Vitamin Mix V10001 | 10 | 40 | 10 | 40 |
| Choline Bitartrate | 2 | 0 | 2 | 0 |
| FD&C Yellow Dye #5 | 0.05 | 0 | 0 | 0 |
| FD&C Blue Dye #1 | 0 | 0 | 0.05 | 0 |
| Total | 1055.05 | 4057 | 773.85 | 4057 |

### Study Design

Nineteen weeks after the beginning of the study, 5 mice in each group (control diet and HFD) were weighed and subjected to an intraperitoneal insulin sensitivity test (IPIST, the protocol of which is detailed below) to check the insulin resistance of the mice. At week 20, glycaemia tests, the protocol of which is detailed below, were performed. Just after the end of these tests, mice received daily 10⁹ CFU of Lactobacillus acidophilus NCFM/mouse/day by oral gavage, for 3 weeks. At week 23, animals were sacrificed after weighing and after determination of glycaemia.

All samples were analysed in a blind manner.

### IPIST Test Protocol

Mice were fasted for 16 hours and received an intraperitoneal injection of human recombinant insulin (0.5 International Units/kg body weight); Actrapid®, Novo-Nordisk, Bagsvaerd, Denmark). Blood glucose was assayed immediately before and at 15, 30, 45, and 60 minutes after injection using a digital glucometer (Medisense Optium, Abbott, Rungis, France).

### Results

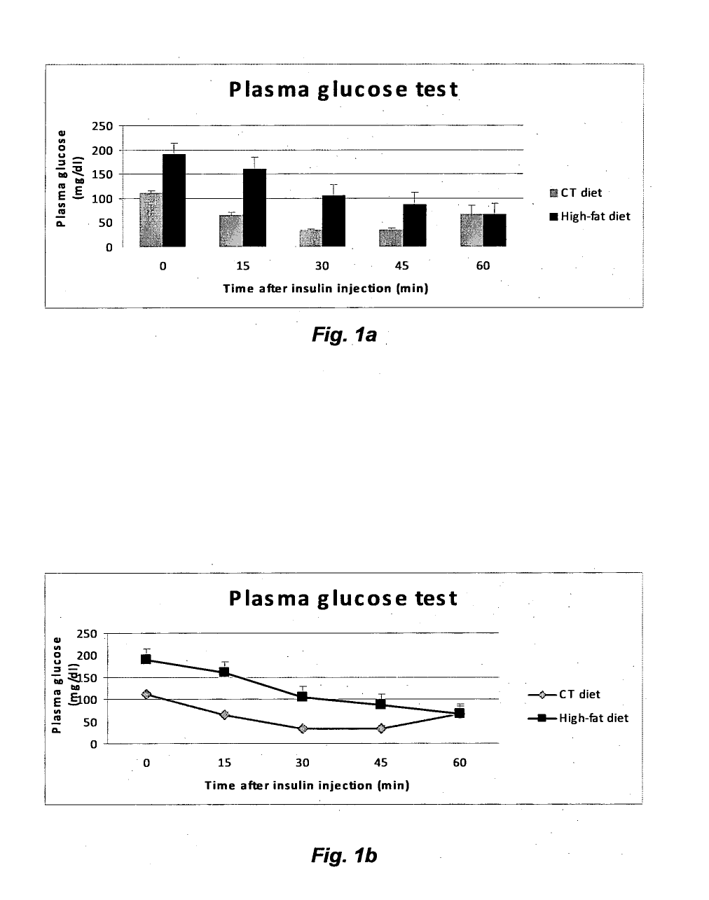

The results of the IPIST test are set out in Table 2 below and in FIGS. 1a and 1b.

TABLE 2

| | | Day | | | | |
|---|---|---|---|---|---|---|
| | | 0 | 15 | 30 | 45 | 60 |
| | Mouse | | | | | |
| Control diet | CT1 | 98 | 82 | 41 | 36 | 35 |
| | CT2 | 114 | 54 | 33 | 28 | 71 |
| | CT3 | 116 | 55 | 31 | 25 | 76 |
| | CT4 | 123 | 74 | 38 | 33 | 32 |
| | CT5 | 108 | 62 | 28 | 50 | 121 |
| High-fat diet | HF1 | 190 | 151 | 97 | 106 | 80 |
| | HF2 | 182 | 162 | 117 | 95 | 71 |
| | HF3 | 207 | 157 | 93 | 92 | 72 |
| | HF4 | 187 | 126 | 99 | 76 | 52 |
| | HF5 | 187 | 209 | 121 | 71 | 56 |
| | Diet | | | | | |
| Mean | Control diet | 112 | 65 | 34 | 34 | 67 |
| | High-fat diet | 191 | 161 | 105 | 88 | 66 |
| Standard error | Control diet | 5 | 6 | 3 | 5 | 18 |
| to mean (SEM) | High-fat diet | 5 | 15 | 6 | 7 | 6 |

Glycaemia of the control group fasted for 16 hours is 112 mg/dl, which is a normal level. In the same group addition of 0.5 IU/kg of insulin lead to a rapid decrease of glycaemia that reach 34 mg/dl after 30 minutes (around 70% of decrease). In the high-fat diet group, fasted glycaemia is 191 mg/dl almost 2 times higher than the normal level indicating a glucose intolerance. After insulin injection, the glycaemia decreases and reach 105 mg/dl after 30 minutes (around 45% decrease) indicating a moderate insulin resistance. This glucose intolerance and this moderate insulin resistance prove that these HFD mice were diabetic.

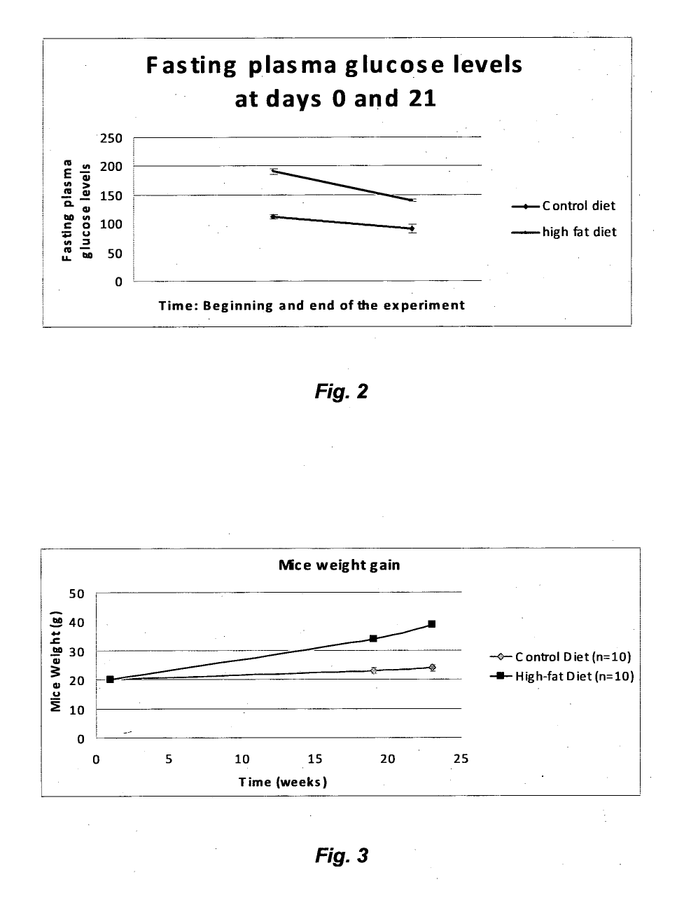

The results for sugar level decrease are shown in FIG. 2; weight increase in the same study is shown in FIG. 3 and Table 3 below.

TABLE 3
Weight of tested mice (g; mean ± SEM)

| Week | 1 | 19 | 23 |
|---|---|---|---|
| Control Diet | 20 ± 1 | 23 ± 1 | 24 ± 1 |
| (n = 10) | | | |
| High-fat Diet | 20 ± 1 | 34 ± 2 | 39 ± 2 |
| (n = 8) | | | |

According to FIG. 2, the administration of Lactobacillus acidophilus NCFM shows a decrease of the fasting blood glucose level in both 2 sets of mice. For the mice fed with the high-fat diet, the decrease of the fasting glucose level is of significance (it allows a decrease of 26.7% in 3 weeks—see FIG. 2+the figures in the Excel to document).

This decrease of glucose level is not the consequence of a weight loss. Indeed, it can be seen from FIG. 3 and Table 3 that the high-fat diet group of mice has doubled its weight after NCFM treatment (from around 20 g to about 39 g) whereas the control group of mice have a very small increase of weight (of around 4 g). This therefore demonstrates that the surprising effect of the microorganisms of the present invention is not linked to a weight loss mechanism.

The above results therefore show that, after administration of Lactobacillus acidophilus NCFM according to the present invention, high-fat diet-induced obese and diabetic mice have a decreased fasting plasma glucose level despite having gained weight while continuing to ingest a high-fat diet.

### Example 2

### Measurement of Adipose Tissue Inflammation

### Methods

### Inflammatory Status of Adipose Tissue Macrophages

The inflammation status of adipose tissue macrophages was measured by first isolating stroma vascular fraction cells (SVF) and then determining the inflammatory and tolerogenic status of the cells with specific cell membrane markers.

The mouse model used in the study of Example 1 was also used for this study.

### Isolation of the Stroma Vascular Fraction (SVF)

Cells were isolated according to Bjôrntorp et al. (J. Lipid Res. 1978, 19, 316-324) with minor modifications. The graft fat pads were digested at 37° C. in phosphate buffered saline (PBS) containing 0.2% bovine serum albumin and 2 mg/ml collagenase for 30 minutes (collagenase A, Roche Diagnostics, Meylan, France). After elimination of undigested fragment by filtration through 25 μm filters, adipocytes fraction were separated from the pellets of the stroma-vascular fraction (SVF) by centrifugation (600×g, 10 min). SVF cells were incubated for 5 minutes in hemolysis buffer (140 nmol/L NH₄Cl and 20 mmol/L tris(hydroxymethyl)aminomethane (Tris), pH 7.6) to eliminate red blood cells and washed by centrifugation in PBS. The number of isolated SVF cells was then counted with counter cell (Coulter Z2). SVF cells were either used for flow cytometry analyses or plated in vitro.

Cell Phenotyping with FACS

Cells isolated from the adipose depots were analyzed by flow cytometry (FACS). Freshly-isolated SVF cells were stained in staining buffer consisting of phosphate-buffered saline containing 0.5% new calf serum and FcR Block reagent (StemCell Technologies, Vancouver, Canada). Cells were incubated with anti-mouse monoclonal antibodies (mAb) or rat immunoglobulins (isotypes) conjugated with FITC, phycoerythrin, PerCP, or allophycocyanin for 30 minutes at 4° C. Triple or quadruple staining was performed by incubating cells with FITC, phycoerythrin, PerCP, and allophycocyanin-conjugated primary antibodies in one step, to precisely characterize the different cell populations. Cells were washed in staining buffer and then analyzed on a FACS (FACS Calibur, Becton Dickinson, Mountain View, Calif.). Data acquisition and analysis were then performed (Cell Quest Pro software, Becton Dickinson).

### Results

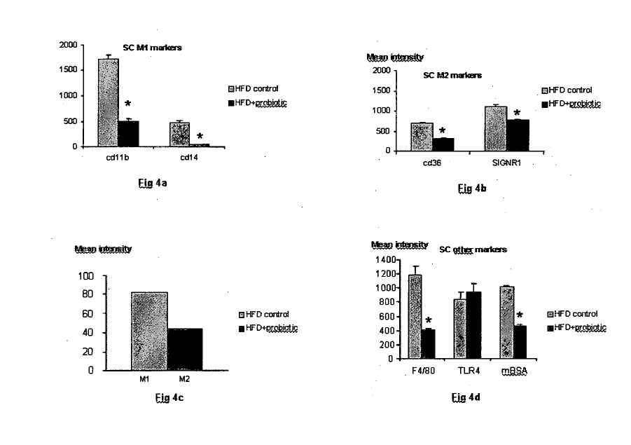

The results are shown in FIGS. 4a to 4d. Mice treated with Lactobacillus acidophilus strain NCFM according to the present invention exhibited lower adipose tissue macrophage inflammation, as measured by reduced Type 1/Type 2 macrophage marker intensity. The dramatic changes in the macrophage phenotype suggest that the cells have become more tolerogenic.

In conclusion, the probiotic treatment results in reduction of inflammatory tone of macrophages and improved tolerance against antigenic factors related to impaired metabolic state.

### Example 3

### Materials and Methods

### Animal Model and Probiotic Treatment

A cohort of fifty C57BI/6 10-wk-old male mice were fed a Normal Chow (NC) (A03, SAFE, Augy, France), or a high-fat diet (HFD) (comprising 72% fat (corn oil and lard), 28% protein and <1% carbohydrates) (SAFE, Augy, France) for 4 weeks. This diet has the peculiar advantage to induce diabetes before the onset of obesity (see for example Cani et al. 2008 “Role of gut microflora in the development of obesity and insulin resistance following high-fat diet feeding”. Pathol Biol (Paris); Cani et al, Diabetes 2008, 57, 1470-81; Knauf et al. Endocrinology 2008, 149, 4768-77; Cani et al., Diabetologia 2007, 50, 2374-83; Cani et al; Diabetes 2007, 56, 1761-1772 and Turini et al. Swiss Med Wkly 2007, 137, 700-4).

The mice underwent an intraperitoneal glucose tolerance test. The area under curve was calculated and the mice dispatched homogeneously according to the different experimental groups or ten mice per group (10 mice per group). The mice were fed four more weeks with a normal chow (n=10) or a HFD (n=40). The HFD mice were treated daily for 4 weeks as follows with vehicle or with Lactobacillus acidophilus NCFM (NCFM) (10⁹/bacteria per mouse). An intraperitoneal test was then performed as described below. The mice were housed in a controlled environment (inverted 12-hour daylight cycle, light off at 10:00 a.m.).

### Plasma Insulin

Insulin concentration was measured from plasma in fasted state as well as in fed state.

### Inflammatory Markers (Real-Time Quantitative PCR)

The inflammation status of adipose, liver and muscle tissue was measured by measuring the concentration of inflammatory markers TNFα, IL-1β, PAI-1, IL6 mRNAs by quantitative RT-PCR analysis. Total mRNAs from the grafted fat pads and the recipient subcutaneous adipose, liver and muscle tissue were extracted using TriPure reagent (Roche, Basel, Switzerland). PCRs were performed using an AbiPrism 7900 Sequence Detection System instrument and software (Applied Biosystems, Foster City, Calif., USA, as described in Cani et al. Diabetes 2007, 56, 1761-1772). The concentration of each mRNA was normalized for RNA loading for each sample using RPL19 rRNA as an internal standard.

### Results

### Plasma Insulin Concentrations

Plasma insulin concentration was assessed in the fasted and the fed state. The data show that, in the fed state, NCFM treatment improved glucose insulin secretion.

Of significance is that high levels of insulin are observed in the fed state of healthy, non-diabetic subjects. Statistically significant results were achieved using NCFM.

### Liver Tissue Inflammation

The results show that, when considering all cytokine mRNA concentrations, HFD induced inflammation in liver tissues. NCFM treatment had a clear anti-inflammatory effect on the liver tissue.

### Muscle Tissue Inflammation

The results show that inflammation was induced by high fat diet also in muscle tissues, although the induction of inflammation was not as strong as in adipose tissue. NCFM treatment tended to lower muscle tissue inflammation.

### Adipose Tissue Inflammation

The results show that the high fat diet clearly induced inflammation in subcutaneous adipose tissue. Treatment with NCFM showed an effect on tissue inflammation.

Taken together, probiotic bacteria showed broad anti-inflammatory effect, with most pronounced effects in adipose tissue and liver tissue.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1-69. (canceled)

70. A method of lowering tissue inflammation in a mammal, comprising administering to said mammal a bacterium of the species Lactobacillus acidophilus.

71. A method according to claim 70, wherein the tissue inflammation is adipose tissue inflammation.

72. A method according to claim 70, wherein the tissue inflammation is liver tissue inflammation.

73. A method according to claim 70, wherein the tissue inflammation is muscle tissue inflammation.

74. A method of reducing the blood glucose level in a diabetic and/or obese mammal without a concomitant decrease in weight gain of said mammal, comprising administering to said mammal a bacterium of the species Lactobacillus acidophilus.

75. A method of reducing insulin resistance in a diabetic and/or obese mammal without a concomitant decrease in weight gain of said mammal, comprising administering to said mammal a bacterium of the species Lactobacillus acidophilus.

76. A method of treating diabetes in a mammal without a concomitant decrease in weight gain of said mammal, comprising administering to said mammal a bacterium of the species Lactobacillus acidophilus.

77. A method of decreasing the metabolic consequences of diabetes in a diabetic and, optionally, obese mammal, comprising administering to said mammal a bacterium of the species Lactobacillus acidophilus.

78. A method of improving glucose tolerance in a mammal, comprising administering to said mammal a bacterium of the species Lactobacillus acidophilus.

79. A method of increasing fed insulin secretion in a mammal, comprising administering to said mammal a bacterium of the species Lactobacillus acidophilus.

80. A method according to claim 70, wherein the mammal is diabetic and obese.

81. A method according to claim 70, wherein the mammal is diabetic and non-obese.

82. A method according to claim 70, wherein the mammal is non-diabetic and obese.

83. A method according to claim 70, wherein the condition is diet-induced and/or diet-associated.

84. A method according to claim 70, wherein the mammal continues to gain weight during the course of the treatment.

85. A method according to claim 70, wherein the mammal continues to ingest a high-fat diet during the course of the treatment.

86. A method according to claim 70, wherein the bacterium is Lactobacillus acidophilus strain NCFM (ATCC PTA-4797).

87. A method of treating cardiovascular disease in a mammal, comprising administering to said mammal a bacterium Lactobacillus acidophilus strain NCFM (ATCC PTA-4797).

88. A method of treating metabolic syndrome in a mammal, comprising administering to said mammal a bacterium Lactobacillus acidophilus strain NCFM (ATCC PTA-4797).
